(19) United States
(12) Patent Application Publication
(10) Pub. No.: US 2025/0126437 A1
(43) Pub. Date: Apr. 17, 2025

(54) Wearable Device and Method for Location Tracking of a User

(71) Applicant: Salvador Calzada, Ennis, TX (US)

(72) Inventor: Salvador Calzada, Ennis, TX (US)

(21) Appl. No.: 18/910,576

(22) Filed: Oct. 9, 2024

Related U.S. Application Data
(60) Provisional application No. 63/589,339, filed on Oct. 11, 2023.

(51) Int. Cl. H04W 4/029; A61B 5/00; A61B 5/11; G04G 21/02; H04W 4/021; H04W 4/90

(52) U.S. Cl. CPC H04W 4/029; A61B 5/1112; A61B 5/6831; G04G 21/02; H04W 4/021; H04W 4/90

(57) ABSTRACT

A wearable location monitoring and tracking device designed to track the location of the wearer and provide alerts to a remote monitor unit. The wearable device includes a housing with electronic components, a band for securing the device, an SOS button for sending alerts, LEDs for status indication, a vibration module, a location module, a wireless communication module, a memory, and a speaker. The remote monitor unit has a monitoring application to track the device's real-time location and to generate notifications. The wearable device sends location information to the remote monitor unit when the SOS button is activated. The method involves pairing the devices, detecting and transmitting location data, generating alerts, and displaying real-time information on a digital map within the monitoring application.

### CROSS-REFERENCE TO RELATED APPLICATION

The present application claims priority to, and the benefit of, U.S. Provisional Application No. 63/589,339, which was filed on Oct. 11, 2023, and is incorporated herein by reference in its entirety.

### FIELD OF THE INVENTION

The present invention generally relates to the field of location tracking systems and devices. More specifically, the present invention relates to a novel wearable device with integrated location tracking. The device includes an SOS button that enables the wearer to ping a monitoring application installed in a remote-control unit to alert others of their location. Parents and caretakers can consistently monitor the wearer's location using the application. Accordingly, the present disclosure makes specific reference thereto the present invention. Nonetheless, it is to be appreciated that aspects of the present invention are also equally applicable to other like applications, devices, and methods of manufacture.

### BACKGROUND

By way of background, children or the elderly individual individuals can wander too far away from home or a designated area and may be unable to come back to their respective places on their own. Children or the elderly individual may not always remember their house address and in such situations even other individuals may be unable assist them in reaching back their homes. Individuals suffering from dementia or memory loss may be unable to go back to their house or any designated area, thereby leaving their caretakers worried and upset. Without a proper tracking or monitoring and tracking system, children or the elderly individual may be unsafe in their surroundings and can end up being in custody of abductors. Children may be kidnapped while going to school or coming back to their home, and their parents or caretakers may be left clueless about their existence. Without any knowledge about a child's location and their existence, the parents may be left in a tragic situation. Parents, caretakers, and guardians may be unaware of where the person or child has wandered off to and may seek assistance of police and/or other security forces. Police may use information from others and may take a lot of time and effort to track a lost individual, that can be extremely dangerous for the lost child or the elderly individual with memory loss.

Further, individuals who are hard of hearing may be unable know when they are called by others while being lost in crowded area or on their way to home or other places. Individuals suffering from hearing loss or hard of hearing individuals may not be able to know when their parents, guardians, or caretakers are trying to communicate with them, thereby leaving their parents/caretakers confused and frustrated.

To overcome such situations, individuals may leave emergency contact cards with the child, elderly, or hard of hearing individuals, while they are away, so that other individuals can assist them in coming back to their homes, however, in such scenarios, the parents/caretakers cannot track their lost child or the elderly individual individuals who has wandered off and may be extremely worried until the lost child or the elderly individual reaches back home.

Therefore, there exists a long felt need in the art for children/elderly location monitoring and tracking system that enables the parents or caretakers to track the location of any child or an elderly individual while they are away from home. There is also a long felt need in the art for a tracking device that enables the parents/caretakers to find and locate the child or the elderly individual who have wandered off and prevents them from being abducted. Additionally, there is a long felt need in the art for a tracking device that ensures the safety of children, elderly with dementia or memory loss, or hard of hearing individuals, or others and prevents them from becoming lost. Moreover, there is a long felt need in the art for a monitoring and tracking device that ensures peace of mind for parents while their children go to school or come back home by themselves. Further, there is a long felt need in the art for children/elderly monitoring and tracking system that can be quickly used to locate the lost individual in case of any emergency. Furthermore, there is a long felt need in the art for a monitoring and tracking system that eliminates the need to seek assistance from police or any other rescue team for locating lost individuals. Finally, there is a long felt need in the art for a communication tool that can be conveniently used to connect lost individuals and their parents/caretakers.

The subject matter disclosed and claimed herein, in one embodiment thereof, comprises a location monitoring and tracking system for a wearer of a wearable location monitoring and tracking device. The system includes the wearable location monitoring and tracking device including a housing adapted to store electronic components, a band connected to the housing, configured to secure the device around a body part of a wearer, an SOS button operable to send an alert signal. The system also includes a remote monitor unit, a software monitoring application is installed in the remote monitor unit, the application is configured to provide a user interface for monitoring the real-time location of the wearable location monitoring and tracking device, display real-time location information on a digital map, and generate notifications when the SOS button is activated or when the wearable location monitoring and tracking device goes beyond a pre-set geofence.

In this manner, the wearable location monitoring and tracking system of the present invention accomplishes all of the forgoing objectives and provides users with a novel wearable device that provides real-time location tracking of the wearer, enabling parents and guardians to monitor the whereabouts of children, elderly individuals, or vulnerable persons. The SOS button of the device enables immediate alert transmission in emergency situations, ensuring rapid response and assistance. The remote monitor unit is equipped with a user-friendly application and enables continuous monitoring and management of multiple wearable devices.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a wearable location monitoring and tracking device. The device comprises a housing, a band connected to the housing and configured to secure the device around a body part of a wearer, an SOS button operable to send an alert signal to a paired remote device, a plurality of LEDs is disposed on the housing and configured to illuminate in different colors to indicate different statuses, a control board is disposed within the housing and comprising electronic components for the functioning of the device, a wireless module is configured to establish a wireless communication channel with the paired remote device, a location module is configured to detect the location of the device, and a memory configured to store geofence information and unique identifier of the device.

In yet another embodiment, a location monitoring and tracking system for a user is disclosed. The system includes a wearable location monitoring and tracking device including a housing adapted to store electronic components, a band connected to the housing, configured to secure the device around a body part of a wearer, an SOS button operable to send an alert signal. The system also includes a remote monitor unit, a software monitoring application is installed in the remote monitor unit, the application is configured to provide a user interface for monitoring the real-time location of the wearable location monitoring and tracking device, display real-time location information on a digital map, and generate notifications when the SOS button is activated or when the wearable location monitoring and tracking device goes beyond a pre-set geofence.

In another embodiment, the wearable location monitoring and tracking device is configured to transmit real-time location data and alert signals to the remote monitor unit via a wireless communication channel and the remote monitor unit is configured to display the received location data and generate notifications based on the alert signals.

In another aspect, the wearable location monitoring and tracking device includes a vibration module disposed within the housing, configured to vibrate the device when the SOS button is activated or when a notification signal is received from the remote device.

In yet another aspect, the LEDs illuminate in a first color when the SOS button is activated and in a second color different from the first color to indicate a low battery status of the device.

In still another embodiment, a speaker of the device is adapted to provide a notification sound when the SOS button is pressed or when an instruction is received from the remote device.

In yet another embodiment, a method for monitoring the location of a wearer using a wearable location monitoring and tracking device and a remote monitor unit is described. The method includes pairing the wearable location monitoring and tracking device with the remote monitor unit via a wireless communication channel, detecting the location of the wearable location monitoring and tracking device using a location module within the device, activating an SOS button on the wearable location monitoring and tracking device to send an alert signal, transmitting real-time location data and the alert signal from the wearable location monitoring and tracking device to the remote monitor unit via the wireless communication channel, and displaying the real-time location information on a digital map within a monitoring application on the remote monitor unit.

Numerous benefits and advantages of this invention will become apparent to those skilled in the art to which it pertains upon reading and understanding of the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of wearer location monitoring wearable device of the present invention in accordance with the disclosed structure;

FIG. 2 illustrates a schematic view showing communication between the wearable location monitoring and tracking device and a remote-control device in accordance with the disclosed structure;

FIG. 3 illustrates a block diagram showing different electronic components and sensors included in the wearable device of the present invention in accordance with the disclosed structure;

FIG. 4 illustrates a perspective view showing location of the wearable device on the application when SOS button is pressed on the wearable device in accordance with the disclosed structure;

FIG. 5 illustrates a flow chart depicting a process of setting up the application for use with the wearable monitoring and tracking device of the present invention in accordance with the disclosed architecture;

FIG. 6 illustrates another flow chart depicting the process of generating notification from the wearable monitoring and tracking device in accordance with the disclosed structure;

FIG. 7 illustrates a perspective view showing waterproof construction of the monitoring wristband of the present invention in accordance with the disclosed structure; and

FIG. 8 illustrates a perspective view showing a wearer wearing the wearable monitoring and tracking device of the present invention in accordance with the disclosed structure.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long felt need in the art for children/elderly location monitoring and tracking system that enables the parents or caretakers to track the location of any child or the elderly individual while they are away from home. There is also a long felt need in the art for a tracking device that enables the parents/caretakers to find and locate the child or the elderly individual who have wandered off and prevents them from being abducted. Additionally, there is a long felt need in the art for a tracking device that ensures the safety of children, elderly with dementia or memory loss, or hard of hearing individuals or others and prevents them from becoming lost. Moreover, there is a long felt need in the art for a monitoring and tracking device that ensures peace of mind for parents while their children go to school or come back home by themselves. Further, there is a long felt need in the art for children/elderly monitoring and tracking system that can be quickly used to locate the lost individual in case of any emergency. Furthermore, there is a long felt need in the art for a monitoring and tracking system that eliminates the need to seek assistance from police or any other rescue team for locating lost individuals. Finally, there is a long felt need in the art for a communication tool that can be conveniently used to connect lost individuals and their parents/caretakers.

The present invention, in one exemplary embodiment, is a wearable location monitoring and tracking device. The device comprises a housing, a band connected to the housing and configured to secure the device around a body part of a wearer, an SOS button operable to send an alert signal to a paired remote device, a plurality of LEDs is disposed on the housing and configured to illuminate when the SOS button is pressed, a control board is disposed within the housing and comprising electronic components for the functioning of the device, a wireless module is configured to establish a wireless communication channel with the paired remote device, a location module is configured to detect the location of the device, and a memory configured to store geofence information and unique identifier of the device.

Reference will now be made in detail to the present preferred embodiments of the invention, examples of which are illustrated in the accompanying drawings. Wherever possible, the same reference numerals are used in the drawings and the description to refer to the same or like parts.

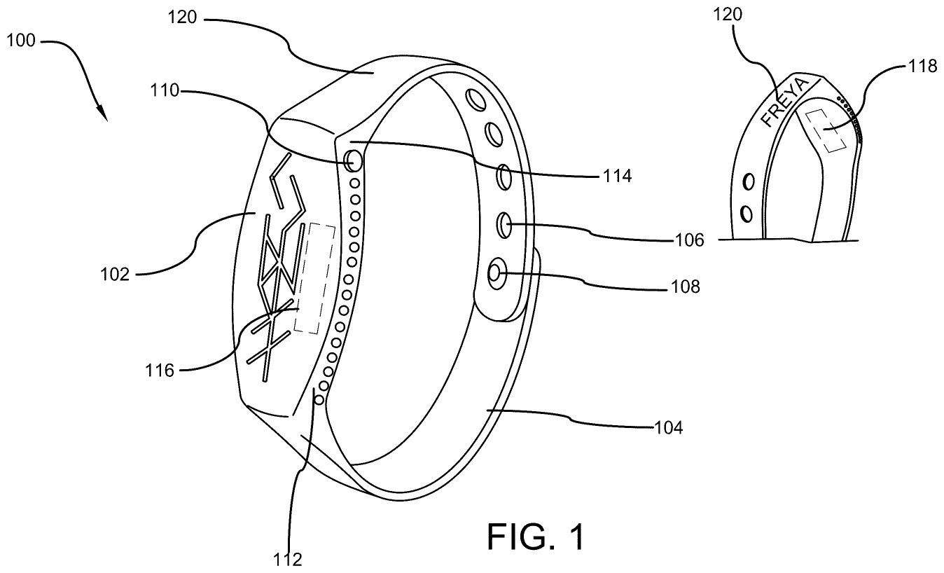

Referring initially to the drawings, FIG. 1 illustrates a perspective view of wearer location monitoring wearable device of the present invention in accordance with the disclosed structure. The wearer location monitoring wearable device 100 of the present invention is designed to monitor location of wearer of the device 100 and to provide an alert to parents, guardians, and caretakers about location of the wearer. More specifically, the location monitoring and tracking device 100 is preferably in the form of a wristband, bracelet, or ankle band but can also be in the form of any wearable device such as a necklace, anklet, armband, and more. The location monitoring wearable device 100 includes a housing 102 adapted to store the electronic components for functioning of the device 100. The housing 102 is preferably designed such that the housing 102 touches or contacts the skin of the wearer when the device 100 is being worn by the wearer or user.

The device 100 includes a band 104 which can be made of metal, rubber, silicone or any other skin-friendly material. The band 104 is used for securing the device 100 around a body part such as wrist or ankle of a wearer and may include a plurality of apertures 106 for receiving and accommodating a clasp 108 to secure the device 100. In different embodiments, the band 104 can be secured using a magnetic fastener, a hook and loop fastener, and the like.

The device 100 includes an SOS button 110 which can be a physical button or a touch button. The SOS button 110, when pushed or touched (i.e., actuated), enables the device 100 to send an alert signal to a paired device such as of a parent, guardian, or caretaker to notify about a location of the device 100. A plurality of LEDs 112 are disposed on the side surface 114 of the housing 102. The LEDs 112 illuminate in a first color when the SOS button 110 is pushed, thereby indicating to the wearer about successful transmission of an alert. The LEDs 112 can vibrate or flash in a second color which is different from the first color to indicate a low battery of the device 100.

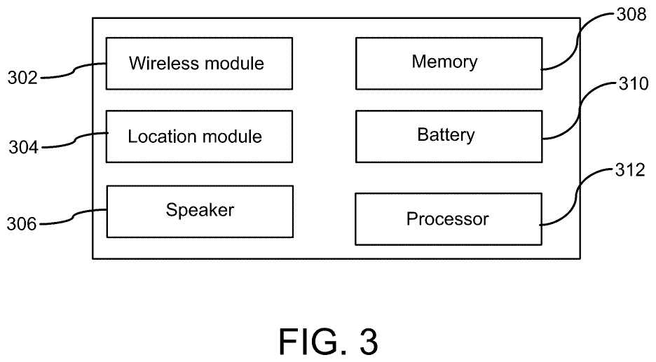

The housing 102 includes a control board 116 which has a plurality of electronic components disposed thereon. The details of the control board 116 are described in FIG. 3. The housing 102 is weatherproof and can have at least IP67 waterproof rating. In some embodiments, the housing 102 is adapted to be open to remove and replace one or more electronic components of the device 100.

A vibration module 118 is disposed in the housing 102 and is adapted to vibrate the device 100 when the SOS button 110 is activated or when a notification signal is sent from a remote or paired device. The vibration module 118 functions as a notification tool for individuals who find it hard to hear. The band 104 has one or more indicia or logos 120 disposed thereon for branding and marketing purposes. The device 100 can come in different sizes, colors, and designs to meet the requirements of different users.

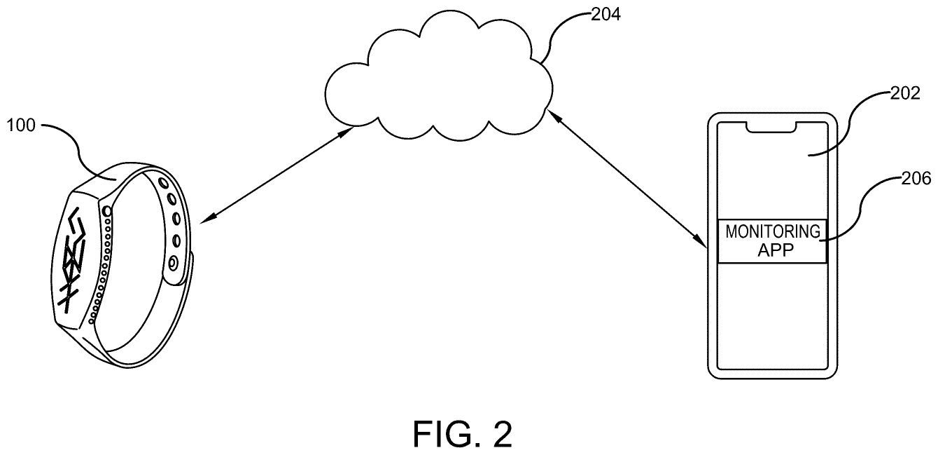

FIG. 2 illustrates a schematic view showing communication between the wearable location monitoring and tracking device and a remote-control device in accordance with the disclosed structure. The wearable location monitoring and tracking device 100 is adapted to communicate with a remote monitor unit 202 which can be in the form of a portable computing electronic device such as a smartphone, smartwatch, tablet, laptop and more. The wearable device 100 is wirelessly coupled with the remote monitor unit 202 via a wireless communication channel 204 which can be any long range or short-range communication channel. Preferably, the communication channel 204 is Bluetooth or any satellite communications channel.

The remote monitor unit 202 includes a monitoring application 206 which is preferably a software application and provides a user interface for monitoring the present location of the coupled wearable device 100. In some embodiments, the application 206 is configured to provide a real-time notification when the SOS button 110 is activated or when the device 100 goes beyond a pre-set geofence using the application 206. In different embodiments, each device 100 is uniquely identified by an identifier which can be any combination of numbers and letters. The application 206 can monitor a plurality of wearable devices and each device is uniquely identified by the corresponding identifier.

FIG. 3 illustrates a block diagram showing different electronic components and sensors included in the wearable device of the present invention in accordance with the disclosed structure. The control board 116 includes the majority of the electronic components but depending on the design and manufacturing technologies, the electronic components and sensors can also be individually and independently included in the device 100. A wireless module 302 is adapted to establish wireless communication channel 204 for communication with the remote monitor unit 202. The wireless module 302 is adapted to provide one or both long range and short-range communication channels. The wireless module 302 enables a user to track the wearable device 100 in real-time.

A location module 304 is adapted to detect the location of the device 100 using one or more of GPS systems, cellular towers, and wireless networks including Wi-Fi networks. The location module 304 can also be adapted to provide indoor location using beacons and is helpful in locating the device 100 inside a premises such as a big building complex. A speaker 306 is included and is adapted to provide a quick notification sound when the SOS button is pressed or when an instruction is received from the remote monitor unit 202. The speaker 306 can be any piezoelectric or mechanical speaker.

A memory 308 is adapted to store unique identifier of the device 100 and is also configured to store geofence information of the device 100. The memory 308 can be any volatile or non-volatile memory and real-time location information of the device 100 may be updated in the memory 308. A button battery 310 is included in the device 100 and it provides electric power for operation of the device 100. The button battery 310 is replaceable and can be recharged wirelessly. A processor 312 executes instructions stored in memory device 308 such as one or more programs implementing process. The processor 312 may perform a function based on signals received from the remote monitor unit 202 and from the location module 304.

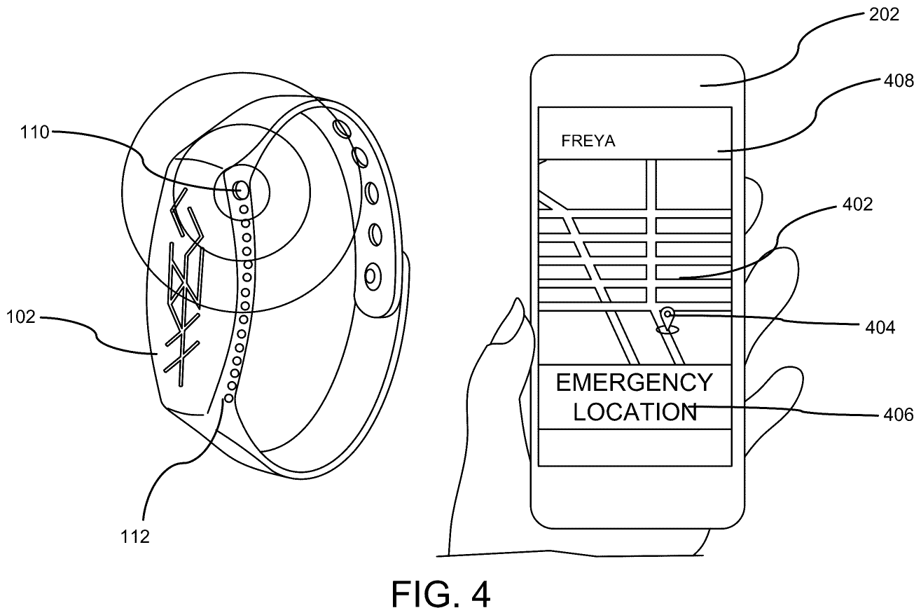

FIG. 4 illustrates a perspective view showing location of the wearable device on the application when SOS button is pressed on the wearable device in accordance with the disclosed structure. As illustrated, when the SOS button 110 is activated or pressed by the wearer, a wireless instruction including encoded real-time location of the device 100 is transmitted to the paired remote monitor unit 202. The application 206 displays a digital map 402 and mark the real-time location information 404 on the map 402. To indicate the emergency status of the location, an “Emergency location” symbol 406 is also displayed by the application 206. A more information symbol 408 when selected enables the application 206 to display additional information such as unique identifier, wearer information and more of the device 100.

In some embodiments, the real-time location information may be displayed on the application 206 and the emergency location information symbol can be automatically displayed when the SOS button 110 is pressed or activated on the device 100.

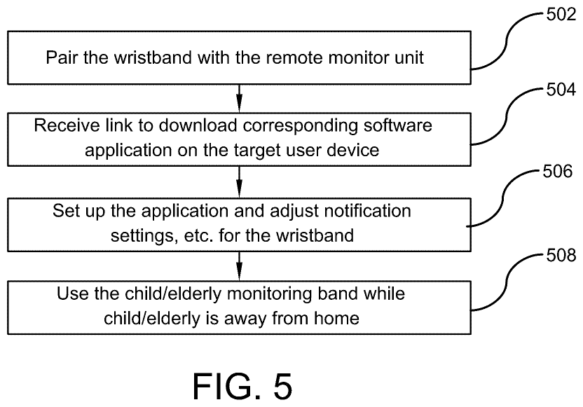

FIG. 5 illustrates a flow chart depicting a process of setting up the application for use with the wearable monitoring and tracking device of the present invention in accordance with the disclosed architecture. Initially, the wearable monitoring and tracking device 100 and the remote monitor unit 202 are paired using the wireless communication channel 204 (Step 502). The pairing can be done by selecting the identifier of the wearable device 100 on the remote monitor unit 202. Once the pairing is complete, then, a download link for downloading the software application 206 is received on the remote monitor unit 202 (Step 504). It should be noted that the link which is in the form of a URL (Uniform Resource Locator) is received after pairing for the first time.

After downloading the application 206, the application 206 is set up and different configurations such as setting up geofence for device 100, notification settings for the SOS button, and more are performed (Step 506). Once the set up is done, then, a child/elderly individual monitoring band 100 is used for tracking location of the wearer of the device 100 using the remote monitor unit 202 (Step 508).

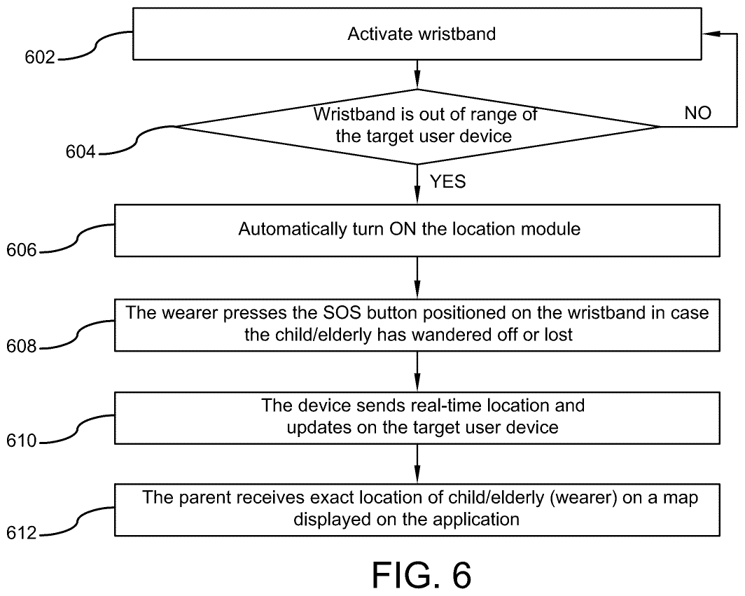

FIG. 6 illustrates another flow chart depicting the process of generating notification from the wearable monitoring and tracking device in accordance with the disclosed structure. Initially, the monitoring wristband 100 is worn and activated by the wearer such as a child or an elderly person (Step 602). Then, it is determined if the monitoring wristband is out of range of the target user device or remote monitor unit (Step 604). In case the wristband is out of the range, then, the location module 304 is automatically activated to track location of the device 100 (Step 606). Then, the SOS button 110 is activated by the wearer in case of an emergency such as when the wearer has wandered off or become lost (Step 608). When the SOS button 110 is pressed, then, the wearable device 100 sends real-time location information to the target user device or remote monitor unit 202 (Step 610). The updated location information is sent in real-time to the user device 202. Finally, the received location information is displayed on the map and a trigger or instruction from the application 206 can also be received by the wearable device 100 (Step 612). Any instruction can be sent from the application 206 to the wearable device 100 and can vibrate and alert the wearer.

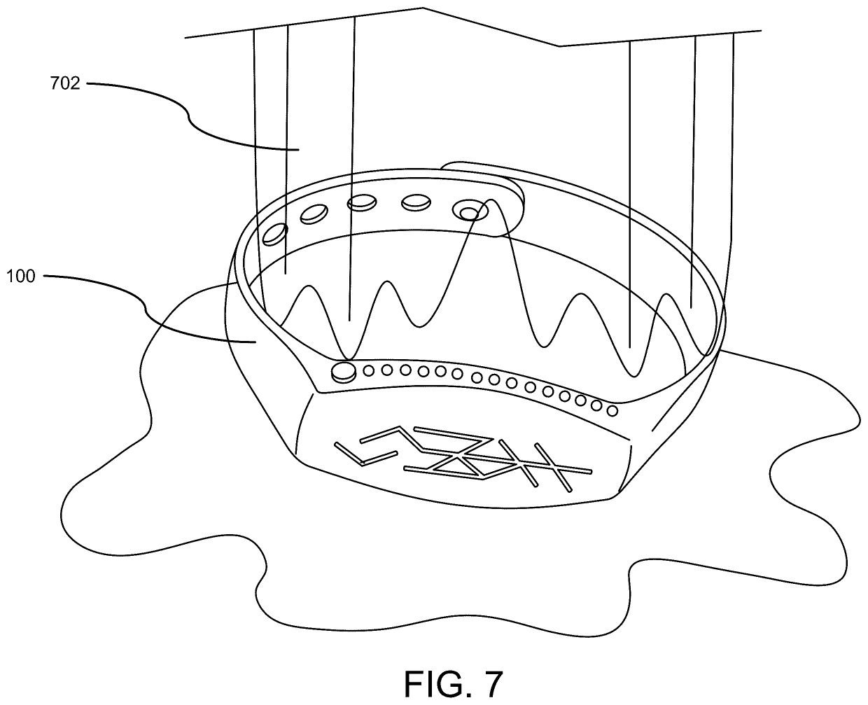

FIG. 7 illustrates a perspective view showing waterproof construction of the monitoring wristband of the present invention in accordance with the disclosed structure. The wearable device 100 has IP67 rating and has a weatherproof construction. The electronic components and sensors may be sealed as well using a waterproof sealing. The wearable device 100 can be worn underwater 702 up to a depth of 40 meters.

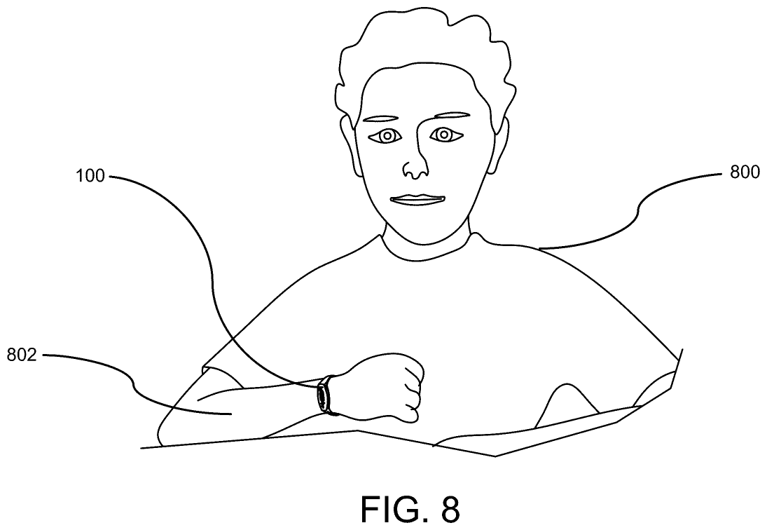

FIG. 8 illustrates a perspective view showing a wearer 800 wearing the wearable monitoring and tracking device of the present invention in accordance with the disclosed structure. The wearable monitoring and tracking device 100 can be worn like a conventional bracelet or watch around the wrist 802. The device 100 in different embodiments can be any type of wearable device which is easily accessible to the wearer for pushing the SOS button.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “monitoring wristband”, “wearable monitoring and tracking device”, “wearer location monitoring wearable device”, “device”, and “wristband” are interchangeable and refer to the wearer location monitoring wearable device 100 of the present invention.

Notwithstanding the forgoing the wearer location monitoring wearable device 100 of the present invention can be of any suitable size and configuration as is known in the art without affecting the overall concept of the invention, provided that it accomplishes the above stated objectives. One of ordinary skill in the art will appreciate that the wearer location monitoring wearable device 100 as shown in the FIGS. are for illustrative purposes only, and that many other sizes and shapes of the wearer location monitoring wearable device 100 are well within the scope of the present disclosure. Although the dimensions the wearer location monitoring wearable device 100 are important design parameters for user convenience, the wearer location monitoring wearable device 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A location monitoring and tracking wearable device comprising:
a band;
a housing including a control board with a plurality of electronic components; and
a paired device;
wherein said housing storing said plurality of electronic components;
wherein said housing contacts the skin of the wearer when said location monitoring and tracking wearable device is worn by a user;
wherein said control board having an SOS button for sending an alert signal to said paired device when actuated;
wherein said paired device is a remote monitor unit possessed by another individual remote from said location monitoring and tracking wearable device;
wherein said another individual is selected from the group consisting of a parent, a guardian, and a caretaker; and
further wherein said remote monitor unit monitoring a location and tracking signal from said location monitoring and tracking wearable device.

2. The location monitoring and tracking wearable device of claim 1, wherein said remote monitor unit selected from the group consisting of a smartphone, a smartwatch, a tablet, and a laptop.

3. The location monitoring and tracking wearable device of claim 2, wherein said location monitoring and tracking wearable device wirelessly coupled with said remote monitor unit through a wireless communication channel.

4. The location monitoring and tracking wearable device of claim 3, wherein said location monitoring and tracking wearable device having a plurality of LEDs disposed on a surface of said housing, wherein said plurality of LEDs illuminate in a first color upon actuation of said SOS button.

5. The location monitoring and tracking wearable device of claim 4, wherein said band having a material selected from the group consisting of a metal, a rubber, and a silicone.

6. The location monitoring and tracking wearable device of claim 5, wherein said band securable around a body part of the user.

7. The location monitoring and tracking wearable device of claim 6, wherein said plurality of LEDs having a second color for low battery indication.

8. The location monitoring and tracking wearable device of claim 7, wherein said first color is different from said second color.

9. The location monitoring and tracking wearable device of claim 6, wherein said housing is weatherproof including at least IP67 waterproof rating.

10. The location monitoring and tracking wearable device of claim 6, wherein said location monitoring and tracking wearable device selected from the group consisting of a wristband, a bracelet, an ankle band, an anklet, and an armband.

11. The location monitoring and tracking wearable device of claim 10, wherein said plurality of electronic components having a vibration module disposed in said housing adapted to vibrate said location monitoring and tracking wearable device upon actuation of said SOS button.

12. The location monitoring and tracking wearable device of claim 10, wherein said plurality of electronic components having a vibration module disposed in said housing adapted to vibrate said location monitoring and tracking wearable device upon notification signal sent from said paired device.

13. A location monitoring and tracking wearable device comprising:
a band;
a housing;
a control board with a plurality of electronic components; and
a paired device;
wherein said housing storing said plurality of electronic components;
wherein said housing contacts the skin of the wearer when said location monitoring and tracking wearable device is worn by a user;
wherein said control board having an SOS button for sending an alert signal to said paired device when actuated;
wherein said paired device is a remote monitor unit possessed by another individual remote from said location monitoring and tracking wearable device;
wherein said another individual is selected from the group consisting of a parent, a guardian, and a caretaker;
wherein said remote monitor unit monitoring a location and tracking signal from said location monitoring and tracking wearable device;
wherein said remote monitor unit having a monitoring application and user interface for monitoring a present location of said location monitoring and tracking wearable device;
wherein said monitoring application having a pre-set geofence area around said paired device; and
further wherein said alert signal actuated when said location monitoring and tracking wearable device is beyond said pre-set geofence area.

14. The location monitoring and tracking wearable device of claim 13 further comprising a plurality of said location monitoring and tracking wearable devices, wherein each of said plurality of said location monitoring and tracking wearable devices having a unique identifier.

15. The location monitoring and tracking wearable device of claim 13, wherein said remote monitor unit selected from the group consisting of a smartphone, a smartwatch, a tablet, and a laptop.

16. The location monitoring and tracking wearable device of claim 2, wherein said location monitoring and tracking wearable device wirelessly coupled with said remote monitor unit through a wireless communication channel.

17. A method of location monitoring and tracking with a wearable device, the method comprising the steps of:
providing a location monitoring and tracking wearable device having a band and a housing including a control board with a plurality of electronic components and a paired device, wherein said housing storing said plurality of electronic components, wherein said control board having an SOS button for sending an alert signal to said paired device when actuated, further wherein said paired device is a remote monitor unit possessed by another individual remote from said location monitoring and tracking wearable device;
selecting said another individual from the group consisting of a parent, a guardian, and a caretaker;
monitoring a location and tracking signal from said location monitoring and tracking wearable device with said remote monitor unit, wherein said remote monitor unit having a monitoring application and user interface for monitoring a present location of said location monitoring and tracking wearable device; and
setting a geofence area around said paired device with said monitoring application.

18. The method of location monitoring and tracking with a wearable device of claim 17, wherein said alert signal actuated when said location monitoring and tracking wearable device is beyond said pre-set geofence area.

19. The method of location monitoring and tracking with a wearable device of claim 18, wherein said remote monitor unit selected from the group consisting of a smartphone, a smartwatch, a tablet, and a laptop.

20. The method of location monitoring and tracking with a wearable device of claim 19, wherein said location monitoring and tracking wearable device wirelessly coupled with said remote monitor unit through a wireless communication channel.
